(19) United States
(12) Patent Application Publication
(10) Pub. No.: US 2017/0326165 A1
(43) Pub. Date: Nov. 16, 2017

(54) USE OF RIBOSE IN FIRST RESPONSE TO ACUTE MYOCARDIAL INFARCTION

(71) Applicant: BIOENERGY, INC., Ham Lake, MN (US)

(72) Inventors: John A. St. Cyr, Coon Rapids, MN (US); David J. Perkowski, San Clemente, CA (US)

(21) Appl. No.: 15/497,421

(22) Filed: Apr. 26, 2017

Related U.S. Application Data
(63) Continuation of application No. 12/384,282, filed on Apr. 2, 2009, now abandoned.
(60) Provisional application No. 61/204,658, filed on Jan. 9, 2009.
(60) Provisional application No. 61/072,774, filed on Apr. 2, 2008.

(51) Int. Cl. A61K 31/7004; A61K 33/00; A61K 31/616; A61K 45/06; A61K 9/00

(52) U.S. Cl. CPC A61K 31/7004; A61K 45/06; A61K 9/0019; A61K 31/616; A61K 33/00

(57) ABSTRACT

D-ribose is administered to patients suffering an acute myocardial infarction during first response care, in order to prevent cardiac compromise. In those patients able to ingest fluids, two to five grams of D-ribose is administered orally. In a patients unable to ingest fluids, or in a patient with and intravenous line, pyrogen-free D-ribose is administered intravenously at a rate of 50-300 mg/kg/hour.

### RELATED APPLICATIONS

This application is related to and claims priority of U.S. Provisional Patent Application Ser. No. 61/072,772, filed Apr. 2, 2008 and U.S. Provisional Patent Application Ser. No. 61/204,658, filed Jan. 9, 2009.

### BACKGROUND OF THE INVENTION

It is well known that the pentose sugar ribose is important in the energy cycle as a constituent of adenosine triphosphate (ATP) and nucleic acids. It is also well known that ribose is found only at low concentrations in the diet, and that further, the metabolic process by which the body produces ribose, the pentose phosphate pathway, is rate limited in many tissues.

Ribose is known to improve recovery of healthy dog hearts subjected to global ischemia at normal body temperatures, when administered for five days following removal of the cross clamp. These inventors have previously discovered (U.S. Pat. No. 6,159,942) that the administration of ribose enhances energy in subjects who have not been subjected to ischemic insult. In the case of human patients, by the time cardiac surgical intervention is performed following presentation of a heart attack patient at a hospital, the condition of the heart and the general state of health are both impaired. Morbidity and mortality following myocardial ischemia, more so in an acute crisis, is increased.

Abnormal cardiac function can occur due to a variety of factors. All of the following factors can negatively affect any medical or surgical outcome. Obviously, tissue death contributes to loss of viable myocardium, which ultimately affects myocardial function. Factors such as preload, after-load, heart rate and rhythm also affect cardiac output status. Volume loading and agents to affect after-load status are commonly provided. However, heart rate and rhythm are most innate and not commonly adjusted to help correct any abnormalities.

Physical conditions also contribute to this physiologically compromised state of the heart. For example, intravascular, including intra-arterial, clots potentially evolving into an infarct of muscle, can severely affect subsequent cardiac function in any patient. First response to assist a heart attack patient may be emergency medical technicians, ambulance staff, hospital receiving staff or clinic office staff. Immediately on reaching the patient, an intravenous line is started, one or two 350 mg aspirin tablets and nitrate or other vasodilators are given. An oxygen line, with or without intubation, is put in place. Interim care is directed at dissolving the occluding clot with such agents as streptokinase, urokinase and tissue plasminogen activator (TPA) in order to get immediate relief of the ischemia and initially stabilize the patient. This scenario is commonly found in patients with acute myocardial infarction (AMI). During this anti-thrombic interval, the function of the heart can be and usually is unstable. Until myocardial instability and dysfunction are improved, an increased morbidity and mortality can be found. Not only is immediate myocardial stabilization important, but subsequent continued stabilization with functional myocardial recovery is the goal of any therapy.

The need remains for a method to stabilize MI patients immediately at first response, so that myocardial stability and function can be restored, thus allowing surgical intervention if indicated.

### SUMMARY OF THE INVENTION

It has been discovered that administration of D-ribose will assist in the stabilization of the heart following AMI until other interventions can be instituted. If the patient is able to ingest fluids, a 3% solution is prepared and sipped by the patient until at least ten grams of ribose have been ingested over at least one hour. The administration of ribose is continued for at least one day. When the patient is on intravenous (IV) drip, pyrogen-free D-ribose may be added to the infusion. The preferred dosage of ribose is 50-300 mg/kg/hour administered intravenously. The most preferred dosage of ribose is 200 mg/kg/hr. Most preferably, the patient is coadministered an equimolar amount of Dextrose or 5% w/v Dextrose, given simultaneously with the ribose.

The oral or IV administration of ribose is continued until the patient has attained a degree of myocardial stability. For some patients, no surgical intervention is necessary. For those patients selected for CABG, interest has increased for off-pump cardiac bypass grafting (OCBPG).

If surgical intervention is indicated, as the patient is being prepared for surgery, MgSO₄ is added to the IV drip until the patient has been given an initial five grams of MgSO₄, preferably given in a 100 cc bolus. The levels are monitored to maintain a concentration of 2.5 meq/l during surgery and for the first 24 hours post-surgery. Potassium cation is carefully maintained at 4 meq/l. Preferably, milronine (Primacor, Sanofi-Aventis, Bridgeport, Conn.) at 0.5 mcg/kg/min is administered IV.

A method of preparation of substantially pure, pyrogen-free ribose suitable for intravenous administration is disclosed. The intravenous dosage given of each agent or agents is from 30 to 300 mg/kg/hour, delivered from a solution of from 5 to 30% w/v of pyrogen-free D-ribose in water. When D-glucose is to be co-administered, it may be delivered from a solution of from five to 30% w/v of D-glucose in water. The agent or agents to be administered are tapped into an intravenous line and the flow set to delivered from 30 to 300 mg/kg/hour agent or agents. Most preferably, pyrogen-free D-ribose is administered with D-glucose, each being delivered intravenously at a rate of 200 ma/kg/hour. When the agent or agents are administered orally, from one to 20 grams of D-ribose is mixed in 200 ml of water and ingested one to four times per day. Most preferably, five grams of D-ribose and five grams of D-glucose are dissolved in water and ingested four times per day.

Patients in the intensive care unit (ICU) are administered pyrogen-free D-ribose as a single agent or more preferably in combination with D-glucose. The agent or agents are administered intravenously during the stay in the ICU. The intravenous dosage to be given of each agent or agents is from 30 to 300 mg/kg/hour, delivered from a solution of from 5 to 30% w/v of pyrogen-free D-ribose in water. When D-glucose is to be co-administered, it may be delivered from a solution of from 5 to 30% w/v of D-glucose in water. The agent or agents to he administered are additionally tapped into an intravenous line and the flow set to deliver from 30 to 300 mg/kg/hour agent or agents. Most preferably, pyrogen-free D-ribose is administered with D-glucose, each being delivered at a rate of 100 mg/kg/hour. When patients are released from the ICU, it is beneficial to continue the administration of the agent or agents. Intravenous administration will be continued while an IV line is in place. When the agent or agents are administered orally, from one to 20 grams of D-ribose is mixed in 200 ml of water and ingested one to four times per day. Most preferably, five grams of D-ribose and five grams of D-glucose are dissolved in water and ingested four times per day.

### DETAILED DESCRIPTION OF THE INVENTION

The following examples are given to show how the invention has been or is to be practiced. Those skilled in the art can readily make insubstantial changes in the methods and compositions of this invention without departing from its spirit and scope. In particular, it will be noted that in most of the examples, it is suggested that D-glucose be given along with D-ribose. It should be noted that the administration of D-glucose is advised not as a therapy, but to avoid the hypoglycemia that can occur when D-ribose is given. If it has been determined that a particular patient does not show hypoglycemia on D-ribose administration, the D-glucose may be eliminated.

### Example 1. Preparation of Substantially Pure, Pyrogen-Free Ribose

Products produced by fermentation often have some residue of pyrogens, that is, substances that can induce fever when administered intravenously. Among the most frequent pyrogenic contaminants are bacterial endotoxins. Therefore, endotoxin analysis is used to determine whether a substance is or is not essentially free of pyrogens. Additionally, congeners, that is, undesirable side products produced during fermentation, and heavy metals may be carried through and present in the fermentation product.

D-ribose prepared by fermentation and purified is approximately 97% pure and may often contain low levels of endotoxin. While this product is safe for oral ingestion and may be termed “food grade” it is not “pharma grade,” suitable for intravenous administration. D-ribose may be purified to pharma grade and rendered pyrogen-free. Briefly, all equipment is scrupulously cleaned with a final rinse of pyrogen-free water, which may be double distilled or prepared by reverse osmosis. All solutions and reagents are made up with pyrogen-free water.

A solution of about 30% to 40% ribose in water is prepared. Activated charcoal is added and the suspension mixed at least 30 minutes, while maintaining the temperature at 50-60° C. The charcoal is removed by filtration. The filtered solution should be clear and almost colorless. Ethanol is added to induce crystallization and the crystals allowed to grow for one or two days. For convenient handling, the crystals are ground and transferred to drums, bags or other containers. Each container is preferably supplied with a bag of desiccant. The final product is essentially pure and free of pyrogens, heavy metals and congeners.

Pyrogen-free D-ribose, suitable for intravenous use, is available from Bioenergy, Inc., Ham Lake, Minn.

### Example 2. Previous Results of Administration of D-Ribose to MI Subjects

A. Foker (U.S. Pat. No. 4,719,201) found that healthy dog hearts require up to nine days to re-establish normal baseline ATP levels following a 20 minute, normothermic period of global myocardial ischemia. Administration of D-ribose immediately at reperfusion and continuing for at least four days enhanced ATP recovery. A protocol was devised to test whether human subjects undergoing either valve surgery plus coronary artery bypass graft (CABG) or CABG alone with decreased heart function would benefit from the administration of ribose following heart surgery as did the healthy dogs of the Foker study.

Recently, the use of ribose to precondition rats subjected to an anterior MI was investigated. Significant improvement in some parameters of heart function was found, including LV diastolic diameter, LV systolic diameter, ejection fraction and shortening fraction. Intravenous ribose was administered for 14 days previous to the inducement of MI. It was not reported whether ribose administration was continued during and after the procedure. (Befera, et al., J. Surg. Res. 2007:137(2):156). The early intervention of ribose administration as shown by Befera in healthy, young rats with induced MI may be applicable to middle-aged humans suffering from AMI.

B. A preconditioning study was performed in human patients scheduled for surgery. After FDA and institutional review board approval, informed consent was obtained from 49 patients for enrolment in a prospective single center, double-blind, placebo-controlled clinical trial, designed to evaluate the efficacy of D-ribose for the treatment of myocardial dysfunction resulting from globally induced ischemia during cardiac surgical procedures.

Inclusion criteria were:
Males or females aged 18 or older
Patients with documented coronary artery disease undergoing CABG with an ejection fraction (EF) of 35% based on echocardiography, radionuclide imaging or cardiac catheterization done within eight weeks of surgery. (If more than one method was used to evaluate EF during this period, the mean values of the various methods were 35%).
Patients undergoing single or double valve replacement with documented coronary artery disease also undergoing CABG; or patients undergoing single or double valve replacement without CABG
Serum creatinine of <2.35 mg/dl
For females of childbearing potential, a negative pregnancy test.
Signed consent forms.

The test article, placebo or ribose, was dispensed according to computer-generated randomization schedule either for patients undergoing CABG only or for patients undergoing heart valve surgery +/−CABG. All patients received a high dose narcotic anaesthesia technique consisting of either fentanyl (50-100 μg/kg) or sufentanil (10-20 μg/kg) and midazolam. No restriction was placed on the type of anaesthetic agents administered. The anaesthesiologists and surgeons responsible for the care of the patents made the clinical decision to use inotropic support, intra-aortic balloon pump support or post bypass circulatory support based on their knowledge of patients requirements and accepted medical practice and without regard to test article status. The test article infusion was started intravenously at the time of aortic cross clamping and continued until the pulmonary artery catheters introducer was removed or for five days (120) hours whichever occurred first. The surgeons responsible for the clinical care of the patients removed the pulmonary artery catheter cordis without regard to test article stats.

Hemodynamic measurements consisting of heart rate, blood pressure, pulmonary artery pressures, pulmonary capillary wedge pressure (PCWP), central venous pressure (CVP) and thermodilution cardiac index (CI) were obtained at the following time intervals: immediately prior to induction of anaesthesia, post induction of anaesthesia prior to sternotomy, post sternotomy prior to initiation of cardiopulmonary bypass, upon successful termination of cardiopulmonary bypass prior to sternal closure and prior to reversal of heparinization with protamine, post closure of the sternum, upon arrival in the intensive care unit and at one or two hour intervals until the pulmonary artery a catheter was removed.

Transesophageal echocardiography data (H.P. Sonos OR, 5.0 MHz, Andover, Mass.) was collected at the following time intervals: post induction of anaesthesia prior to sternotomy, and immediately post closure of the sternum. Transthoracic echocardiography (H.P. Sonos 1500. 2.5 MHz, Andover, Mass.) measurements were made on day three and day seven of the study period. For both the transesophageal and transthoracic echocardiograms, the following long axis and short axis mid-papillary area changes were measured in triplicate by acoustic quantification techniques: end diastolic area (EDA), end systolic area (ESA), fractional area change (FAC), +dA/dt and −dA/dt. All area change data were also analyzed by manual off line analysis. EF was also determined off line using a long axis view, In addition, regional wall motion was quantified as the following: normal=1, hypokinetic=2, akinetic=3 and dyskinetic=4. The wall motion index score (WMIS) and percentage normal myocardium were calculated by reading a maximum of sixteen segments. Echocardiography data for evaluating wall motion and area change was analyzed only if greater than 75% of the endocardial border could be visualized through a complete cardiac cycle. Off line analysis was performed on an Image View echocardiography workstation (Nova Microsonics, Allendale, N.J.). Transmitral Doppler flow velocity measurements made at the level of the mitral valve leaflets included early diastolic filling (E), the atrial filling component (A) and the E/A ratio. Valvular insufficiency was evaluated and quantified as none, trace, mild, moderate, or severe. An interpreter blinded to both treatment and outcome analyzed all echocardiogrpahy data.

All concomitant medications given within 24 hours of the test article and up through Day 7 were recorded including indication, time started, time completed and total dose(s). Input (NG, oral and intravenous fluids) and outputs (urine and other fluids) were measured and recorded through Day 7 as available per hospital routine.

Clinical outcome parameters included the following: number of attempts to wean from CPB, time to extubation, time to discharge from the ICU, time to hospital discharge, number and duration of inotropic drugs, use and duration of intraaortic balloon pump support, and survival to to 30 days postoperatively.

Blood glucose levels were determined hourly, after initiation of the study drug infusion, by dextrastix (Accu-Chk III, Boehringer Mannheim Corp. Indianapolis Ind.) using blood from an intraarterial catheter. If the blood glucose level remained stable for 12 hours, then subsequent blood glucose levels were measured every 4 to 6 hours until the study drug infusion was stopped. Other clinical laboratory measurements including complete CBC with differential, platelet count, electrolytes, liver function studies, serum osmolarity, and urinalysis were completed the morning following surgery. Abnormal laboratory tests were repeated as clinically indicated until normal or determined not to be clinically significant.

All data were entered into a Microsoft Excel Spreadsheet (v4.0, Microsoft Corp., Redmond, Wash.). Before unblinding, 100% of the echocardiography data, 20% of the hemodynamic data and 5% of all other data were audited. The entry error rate was less than 0.001%. A detailed statistical analysis plan for evaluation of the demographic, safety, and efficacy data was developed before unblinding of the study. All statistics were computed on JMP software (v3.1 for Windows, SAS Institute Inc., Cary, N.C.). The plan excluded those patients deemed not possible to evaluate because of protocol violations including interruption of test article administration for greater than a four-hour period (one subject), technically limited echocardiographic studies, and interoperative surgical difficulty not related to pharmacological treatment (two subjects). Covariates included age, aortic cross clamp time, baseline EF, and baseline WMIS. Statistical tests included Chi square, t-test, univariate ANOVA for repeated measures, and ANCOVA. For all statistical tests p<0.05 (two-tailed) was considered to represent statistical significance.

After the inclusion of 49 patients, the enrollment f additional patients was suspended because of an institutional decision to extubate all cardiac surgery patients within six hours postoperatively and discharge the patients from the ICU within 24 hours, if clinically stable. This decision required an alteration of anaesthetic technique and postoperative management. As a result of early this termination of the study, we excluded from analysis nine enrolled patients, including those patients with isolated mitral insufficiency (n=3), isolated mitral stenosis (n=3), combined aortic and mitral valve disease (n=3).

The demographic and baseline measurements of cardiac function for those patients for whom both baseline and day 7 EF could be determined by echocardiography and who had aortic stenosis or coronary artery disease (n=27) was examined. The ribose treated patients were older (66.5 yr. vs. 56.4 yr, p=0.026) and tended to have a lower baseline EF than the placebo treated patients. However, the baseline difference in EF did not achieve statistical significance. Other significant baseline differences were not found for these patients.

The mean baseline EF for placebo treated patients declined from 55% to 38% at Day 7 (p=0.0025). The mean baseline and Day 7 EF for the ribose treated patients was unchanged (44% vs. 41%, p=0.49), The split-plot time effects of treatment group on EF as calculated from a univariate ANOVA model for repeated measures with random effect was statistically different (prob >F, p=0.04). EF was maintained in the ribose treated patients whereas in placebo treated patients, EF declined. The hypothesis tests provided by JMP agree with the hypotheses tests of SAS-PROC GLM (types III and IV).

Five patients (28%) in the ribose treated group developed hypoglycemia (fingerstick glucose <70 mg/dl)) a known side effect of this pentose sugar. No placebo treated patients developed hypoglycemia. The mean glucose level in those patients developing hypoglycemia was 58 mg/dl. The lowest glucose level was 31 mg/dl. Three subjects were treated with a bolus injection of D50W; one subject was treated with oral apple juice; one subject did not require treatment. The study drug infusion was stopped in two subjects because of hypoglycemia. None of these patients developed neurological or other clinical symptoms associated with hypoglycemia. There were no statistical differences in the other clinical laboratory measurements. It is important to note that analysis including those subjects who had protocol violations did not alter any statistical outcome.

This study demonstrates the potential benefit of D-ribose infusion at 100 mg/kg/hr for the preservation of postoperative EF in patients who have CABG. Infusion will be more effective than the oral administration of the study, since it can be continuous rather than intermittent and can be administered to patients unable to ingest food or liquids. In the study, the EF decreased from baseline in the placebo treated patients whereas in the ribose treated patients, EF was maintained. It may be noted that although randomization was performed using standard methods, in this population group, the patients receiving ribose had a lower EF. Nonetheless, the EF was maintained while the higher EF of the placebo controls decreased.

### Example 3. Metabolically Directed Protocol

Following the initial study described in Example 2, 366 consecutive patients, 41-88 years of age, undergoing OPCABG were enrolled. Of these, 89 had recent MIs and seven presented with MIs within one to seven days. Prospectively collected data included comorbidities, hemodynamics and outcomes. All patients were managed with a protocol emphasizing normoglycemia, normothermia and reduced inflammation. Group 1 (n=308) received multiple oral doses (5 gram/dose) of D-ribose prior to and following surgery. Group 2 (n=58) were managed with the same metabolic protocol, but did not receive D-ribose. Group 2 were more likely to have undergone emergent OPCABG (9% versus 1%, p<0.001) but Group 1 had a lower average preoperative cardiac index (CI, see table I). Otherwise, both groups had similar preoperative characteristics including ejection fraction (EJ) and Society for Thoracic Surgery (STS) Risk Indices with nonsignificant trends in the increased comorbidities in Group 1.

Group 1 tended toward less time in intensive care (72 versus 87 hours) and toward a lower requirement for IABP (12% versus 21%), but these trends were not significant. Despite poorer preoperative CI, Group 1 tended toward a higher postoperative CI and the increase after surgery was significantly greater in Group 1 (0.8 versus 0.4, p<0.001). Furthermore, 86% of Group 1 demonstrated an increase in CI but only 66% of Group 2 enjoyed an increase in CI after OPCABG (p<0.001). There were three perioperative MIs, no strokes, two patients required hemodialysis, and there was one postoperative death (Group 1).

TABLE 1

| | | | Preop | | | | |
|---|---|---|---|---|---|---|---|
| | Age | Female | MI < 21 | Preop | STS Risk | Preop | Postop |
| | (yrs) | gender | days | EJ % | Index | CI | CI |
| Group 1 | 70 ± 11 | 23% | 21% | 55 ± 12 | 0.029 ± 0.33 | 2.3 ± 0.5 | 3.0 ± 0.7 |
| Group 2 | 69 ± 10 | 22% | 26% | 56 ± 10 | 0.032 ± 0.041 | 2.5 ± 0.6 | 2.8 ± 0.7 |
| p values | 0.423 | 1.00 | 0.566 | 0.634 | 0.533 | 0.004 | 0.100 |

This protocol was associated with very encouraging outcomes following OPCABG in patients with a high frequency of associated comorbidities, including left main disease and recent MI. Despite a significantly lower preoperative CI in Group 1 patients undergoing initial or repeat (n=7% in Group 1 and 5% in Group 2) OPCABG, these patients receiving D-ribose actually demonstrated better postoperative CI, suggesting enhanced myocardial recovery. This study was not randomized with respect to the addition of D-ribose, but our results suggest that a randomized prospective trial with D-ribose is warranted to further explore the beneficial effects of D-ribose administration following ML Particularly, it would be most beneficial to include intravenous administration of D-ribose to patients suffering an MI. It is expected that since some MI patients may not be able to ingest oral D-ribose, intravenous administration will provide even more benefit to patients suffering a recent ML

### Example 4. Administration of D-Ribose on Admission to Hospital Care

A. While these studies are promising, neither replicates the clinical situation of a patient presenting at first response with an acute myocardial infarction, where time is of the essence. In most cases, MI is a spontaneous event, but an MI can be induced during a procedure such as angiogram, angioplasty or dobutamine echocardiography. Such a patient is generally in the process of compromising cardiac function. Table II shows a comparison of the seven acute, first response MI patients to the 308 patients that were preconditioned with D-ribose, described below as the total patients of Table I.

TABLE II
Comparison of first response MI patients
to the total D-ribose patients of Table I.

| | Age | Preop CI | Postop CI | Change |
|---|---|---|---|---|
| Group 1 of Table I | 70 ± 11 | 2.3 ± 0.5 | 3.0 ± 0.7 | +0.07 |
| First response | 74.7 ± 5 | 2.19 ± 0.7 | 2.60 ± 0.4 | +0.41 |

Note that these first response patients were in the process of experiencing the cardiac compromise that follows an MI and that the administration of D-ribose interrupted this compromise, as can be seen by the continuing lower CI in the patients of Table I (group 2) who were not administered D-ribose. It should also be mentioned that these seven patients are included in Group 1 of Table I. With preloading with D-ribose, they were able to maintain and slightly increase their CI in comparison to the total group.

Standard first response procedures include immediate oxygen, aspirin and vasodilator administration, setting up an intravenous line and clot busting. Example 2 demonstrates that administration of D-ribose intravenously during and after cross clamping of the aorta maintains and improves EF compared to administration of D-glucose; that preconditioning with D-ribose before an induced MI or CABG is beneficial. Table II demonstrates that early intervention, even orally administered, may significantly reduce cardiac compromise when D-ribose is added to the standard first response care of an acute MI patient.

B. Clinical study. A single-center, randomized, double-blinded placebo-controlled clinical trial was designed to determine if administration of D-ribose on admission to hospital care could improve the functional parameters of the heart. D-ribose will be administered orally to those patients able to ingest food and water and intravenously to those patients who are able to ingest food and water. The intravenous dosage of D-ribose is from 30 to 300 mg/kg/hour, delivered from a solution of from five to 30% w/v of pyrogen-free D-ribose in water. When D-glucose is to be co-administered, it may be delivered from a solution of from five to 30% w/v of D-glucose in water. The D-ribose is tapped into an intravenous line and the flow set to delivered from 30 to 300 mg/kg/hour. It has been found in many studies that 100 to 200 mg/kg/hour is adequate for maximum D-ribose benefit. When oral administration is possible, from one to 20 grams of D-ribose is mixed in 200 ml of water and ingested one to four times per day. It has been found in many studies that five grams of D-ribose ingested three or four times per day is adequate. With the availability of pyrogen-free D-ribose for intravenous administration, the stabilization and prevention of cardiac compromise seen in Table II can be available to the unconscious or nauseated patient presenting for first response at a hospital or clinic.

Among the parameters studied will be the size of the infarct and the size of the border zones.

We claim:

1. A method comprising the administration of an effective amount of D-ribose to a patient suffering from acute myocardial infarction wherein the patient's cardiac index is maintained or improved.

2. The method of claim 1 wherein the effective amount of D-ribose is administered orally to a patient who is able to ingest food and water and intravenously to a patient who is not able to ingest food and water.

3. The method of claim 2 wherein the effective amount of D-ribose to be administered orally is two to five grams of D-ribose, administered one to four times per day.

4. The method of claim 2 wherein the effective amount of D-ribose to be administered intravenously is 50 to 300 mg/kg/hour and the D-ribose is pyrogen-free.

5. A method comprising the administration of D-ribose as first response for a patient suffering from acute myocardial infarct and presenting at a hospital or clinic.

6. The method of claim 5 further comprising the administration of aspitin, subbuccal nitrate and inhaled oxygen.
